(19) United States
(12) Patent Application Publication
(10) Pub. No.: US 2017/0304177 A1
(43) Pub. Date: Oct. 26, 2017

(54) USE OF ISOSORBIDE ESTER AND N-ACYLATED AMINO ACID DERIVATIVES AS ANTI-AGEING AGENT FOR THE HUMAN SKIN

(71) Applicant: SOCIETE D'EXPLOITATION DE PRODUITS POUR LES INDUSTRIES CHIMIQUES SEPPIC, Paris Cedex 107 (FR)

(72) Inventors: Laetitia CATTUZZATO, Castres (FR); Sandy DUMONT, Caucalieres (FR); Jerome GUILBOT, Castres (FR)

(73) Assignee: SOCIETE D'EXPLOITATION DE PRODUITS POUR LES INDUSTRIES CHIMIQUES SEPPIC, Paris Cedex 07 (FR)

(21) Appl. No.: 15/518,387

(22) PCT Filed: Oct. 16, 2015
(86) PCT No.: PCT/FR2015/052791

§ 371 Date: Apr. 11, 2017

(30) Foreign Application Priority Data
Oct. 24, 2014 (FR) 1460254

(51) Int. Cl. A61K 8/49; A61Q 19/08

(52) U.S. Cl. CPC A61K 8/4973; A61Q 19/08

(57) ABSTRACT

A compound of formula (I) is disclosed, in which R′ and R″, which may be identical or different, represent either a hydrogen atom or a monovalent radical of formula (IIa); or of a composition (C1) including between 99% and 20% by mass of a compound of formula (Ia), and between 1% and 80% by mass of a compound of formula (1b), with the aim of preventing or retarding the appearance of the signs of ageing of the human skin or lips or to eliminate the signs, the use being in the form of a cosmetic composition. Also disclosed is a method using the product of formula (I) or composition (C1).

The present invention relates to the use of esters of N-acylamino acid derivatives and of isosorbide as an antiaging agent for human bodily skin, and also to cosmetic, pharmaceutical or dermo-pharmaceutical compositions for topical use comprising said esters of N-acylamino acid derivatives and of isosorbide intended for preventing aging of human bodily skin.

Human skin constitutes the first image offered with regard to others, and, consequently, improving its appearance is a subject of constant concern for humans. The skin reflects a state of well-being, which is often associated with youth, and, a contrario, with a state of fatigue and/or aging. Aging of the skin is thus a concern for humans and more particularly for consumers of cosmetic products, who are in search of solutions for attenuating and/or preventing the visible manifestations associated with said aging. This aging of the skin is observed in the various skin tissues and is characterized by metabolic, functional, cellular, architectural and tissue impairment, leading to visible external effects characterized by the appearance and increase of wrinkles, by a dull complexion, by a lack of uniformity of the complexion (dischromia), or by a modification of the texture and properties, especially the biomechanical properties, of human bodily skin.

Aging of the skin results, on the one hand, from factors intrinsic to each individual (characteristics of the genetic heritage intrinsic to each individual) and, on the other hand, from environmental factors. Among the environmental factors that may cause aging of skin, mention may be made of repeated and prolonged exposure to sunlight, and more particularly exposure to ultraviolet rays, exposure to atmospheric pollution and to cigarette smoke, various forms of oxidative stress that may result, inter alia, from the factors mentioned previously, and also psychological, emotional and nervous stress. Repeated and prolonged exposure of human skin to solar radiation, and more particularly to ultraviolet rays, leads to a form of aging commonly known as photoaging. This photoaging is well documented in the scientific literature and causes skin impairments at various levels, one of the most commonly known skin impairments being solar elastosis, which is characterized by profound changes in the architecture and organization of the elastic fibers of the dermis. These changes lead to a characteristic aspect of such skin, which shows very deep and marked wrinkles, leading to a leathery, i.e. stiff, crevassed and browned, skin appearance and also to changes in its mechanical properties. The changes in the mechanical properties of human skin, associated with aging, are due to impairment of the dermal extracellular matrix, composed of elastic fibers and collagen fibers, and also to impairment of the cellular characteristics. Schulze et al. (1) have shown that the dermal fibroblasts become stiff with age, having an influence on the cellular functions involving the cytoskeleton, such as the contractile, migratory and proliferative properties, which are important for the reorganization of the extracellular matrix.

It is also known that reactive oxygen species (ROS) in excess in human skin (whether the stimulus is exogenous or the production is endogenous) create irreversible bonds with proteins, identified by the term “carbonyl proteins”, which then lose their function. A link has recently been demonstrated between these carbonyl proteins and their impact on key cellular functions such as carbohydrate metabolism, protein maintenance, cellular mobility, including migration, and protein homeostasis (2) (Baraibar and Friguet, 2013). Studies investigating the effect of human sera, originating from donors of various ages, on the migratory properties of fibroblasts were performed by Kondo et al. (3). The data obtained show that the serum of elderly donors inhibits the migratory properties of fibroblasts, and even those of fetal fibroblasts. This illustrates the importance of the intrinsic factors in the problem of aging of the skin.

The “photodynamic” technique has been described as being particularly suitable for rejuvenating (i.e. reducing the wrinkles and fine lines, pigmentary marks, etc.) “photo-exposed” skin, i.e. skin exposed to solar radiation, and more particularly to ultraviolet rays. The mechanism via which this technique acts has recently been studied (4), and it turns out that its mode of action proceeds especially via an increase in the fibroblast population and also via an increase in the migratory capacity of said fibroblasts.

These recent studies thus show that impairment of the migratory properties of fibroblasts contributes toward the phenomenon of aging of the skin. These migratory properties are important and described in the context of the process of repair of skin lesions. Dysfunctions of this process in the case of the elderly are widely described, thus illustrating the importance of this cellular functionality.

As a result, improving the migratory properties of the dermal fibroblasts of human skin and/or increasing the fibroblast population constitute means for preventing and/or treating aging of human bodily skin, and more particularly for preventing and/or treating the visible effects of said aging, for example wrinkles, dull complexion, lack of uniformity of the complexion (dischromia) and stiffness of human bodily skin, caused by natural aging or by prolonged exposure to sunlight, and more particularly exposure to ultraviolet rays, or by exposure to oxidative stresses.

Physical processes intended for stimulating the migration of human skin fibroblasts are known, and, among these, mention may be made of low-intensity laser irradiation (exposure to a wavelength of 632.2 nm) more particularly intended for patients suffering from diabetes (5). Many pharmaceutical active principles also exist, which stimulate fibroblast migration and which are mainly prescribed for the cicatrization process. Mention may thus be made of recombinant human lactoferrin produced by transgenic rice plans (6). These physical processes and these pharmaceutical active principles are generally used for individuals suffering from a pathology, and are unsuitable for cosmetic uses.

Cosmetic active principles also exist which are plant or bacterial extracts and which are described as acting on fibroblast migration. Mention may thus be made of curcumin extracts, which stimulate cicatrization when used at low doses and act on fibroblast migration at higher dose (7); Pouteria lucuma nut oils, characterized by the majority presence of linoleic acid, oleic acid, palmitic acid, stearic acid and γ-linolenic acid, which are described as stimulating fibroblast migration and vinculin expression, and suitable for accelerating the cicatrization of skin wounds (8); the combination of extracts of Vigna marina, Cocos nucifera L., Terminalia catappa L. and Hibiscus tiliaceus L. present in cosmetic compositions for treating wounds, improving cicatrization and treating age-related skin problems, described in the international patent application published under the number WO 2010/127396 A1. The international patent application published under the number WO 2010/056908 A1 discloses the use of an extract of Pouteria lucuma and more particularly of oils contained in their kernel, for improving the migration of human fibroblasts. The use of plant and bacterial extracts has the drawback of showing unreliable performance over time due to the variability of the content of the starting materials.

Synthetic cosmetic active principles also exist, for example peptides, which are described as acting on fibroblast migration. Mention may thus be made of pentapeptides of formula Lys-Thr-Thr-Lys-X in which X represents any natural amino acid but preferentially serine and of which a fatty acid chain (C2 to C22) is grafted onto the N-terminal amine and/or its esterified carboxyl group, described in the French patent application published under the number FR 2 783 169. These pentapeptides are incorporated into cosmetic or pharmaceutical compositions to bring about an increase in the synthesis of collagen and glycosaminoglycans (by radioactivity) on skin explants and an increase in the proliferation of normal human fibroblasts in culture, and consequently to improve the appearance of the skin in the context of its natural aging; its drying-out and its cicatrization. The international patent application published under the number WO 97/17835 discloses peptides containing at least one sequence of three amino acids (Lys-Lys-Gly, Gly-His-Lys or Glu-His-Lys) conjugated to a monocarboxylic or dicarboxylic acid, incorporated into cosmetic or pharmaceutical compositions as a cicatrizing and antiwrinkle agent, showing an effect on the synthesis of collagen I by fibroblasts.

N-Acylamino acid derivatives are chemical ingredients that are widely used for the preparation of cosmetic, dermocosmetic, dermopharmaceutical and pharmaceutical compositions, on account of their various active properties.

Mention may be made especially of the description of lipophilic N-acylamino acid derivatives in the American patent published under the number U.S. Pat. No. 6,864,250, used in methods for treating wounds and burns. The European patent application published under the number EP 505 868 A1 describes N-acylamino acid derivatives and salts thereof, and also the use thereof for treating osteoporosis and for promoting cicatrization. The Hungarian patent application published under the number HU 57582 describes N-acylamino acid derivatives and salts thereof, and more particularly N-acyl derivatives of cysteine, and also the local use thereof on wounds after application of lidocaine. The Japanese patent application published under the number JP 2002-179518 describes N-acyl glutamic acid and aspartic acid derivatives and salts thereof, characterized by acyl chains comprising from 6 to 24 carbon atoms, used for incorporation into the preparation of cosmetic compositions intended for sensitive, dry and atopic skin types. None of these documents describes the use of N-acylamino acid derivatives for improving the migratory properties of dermal fibroblasts of human skin and/or for increasing the fibroblast population so as to prevent and/or treat aging of human bodily skin.

The European patent application published under the number EP 1 471 881 discloses that N-acyl α-amino acid derivatives and especially N-undecylenoyl phenylalanine, have affinity toward the melanocyte specific hormone (α-MSH) receptor and thus induce lightening of the skin via the following biochemical mechanism: competition between the hormone α-MSH and the molecule with affinity toward the α-MSH receptor leads to a reduced level of binding of said hormone to the cell receptors; this competition has the consequence of inhibiting the activity of adenylate cyclase, which results in reduced transformation of ATP into intracellular cyclic AMP; the reduction in the level of cyclic AMP results in inhibition of the enzyme protein kinase A (PKA); inhibition of protein kinase A results in reduced activation of tyrosinase due to the reduced transformation of the latter into phosphorylated tyrosinase; this reduced activation of tyrosinase results in a reduction in melanin synthesis and hence in reduced pigmentation of the skin.

Japanese patent application number 2000-229121 describes the use of N-acylamino acid polyol esters as efficient surfactants.

The international patent application published under the number WO 2010/034917 describes monoesters and diesters of polyols of N-(ω-undecylenoyl) phenylalanine, and more particularly the monoesters and diesters resulting from the reaction of glycerol and of N-(ω-undecylenoyl) phenylalanine, and the uses thereof as agents for lightening human skin.

The international patent application published under the number WO 2013/001192 A1 describes monoesters and diesters resulting from the esterification reaction between isosorbide and N-acylamino acid derivatives, the uses thereof as cosmetic active agent, for preventing and/or limiting the unesthetic effects generated by hypoxia of endothelial cells of the human body, and more particularly for preventing and/or limiting the unesthetic effects generated by shadows under the eyes, bags around the eyes and/or heavy legs.

In the context of their search for novel cosmetic active agents for preventing and/or treating signs of aging of human skin or the lips, the inventors focused on developing a novel technical solution consisting in using products resulting from the esterification reaction between isosorbide and N-acylamino acid derivatives.

For the purposes of the invention, the term “signs of aging of human skin or the lips” means any change in the outer appearance of the skin or the lips due to aging, whether it be chronobiological and/or photo-induced aging and/or aging resulting from exposure to environmental stresses (atmospheric pollution, contact with hazardous substances), for instance wrinkles and fine lines, impairment of the microrelief, lack of elasticity and/or tonicity of the skin, lack of density and/or firmness of human skin or the lips, but also any internal modification of the skin that is not automatically reflected by a modified outer appearance, for instance any internal degradation of the skin consecutive to exposure to ultraviolet rays.

This is why, according to a first aspect, a subject of the invention is the use of a compound of formula (I):

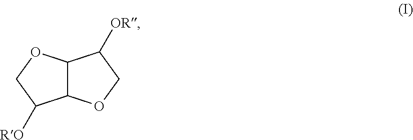

in which R′ and R″, which may be identical or different, represent either a hydrogen atom; or a monovalent radical of formula (IIa):

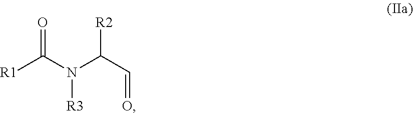

in which formula (IIa) the group R1-C(═O)— is chosen from the radicals octanoyl, decanoyl, ω-undecylenoyl, dodecanoyl, tetradecanoyl, hexadecanoyl, octadecanoyl, 9-octadecenoyl or 9,12-octadecadienoyl, R3 represents a hydrogen atom, and R2 represents a radical chosen from methyl, isopropyl, isobutyl and 1-methylpropyl radicals, it being understood that, in said formula (I), at least one of the radicals R′ and R″ does not represent a hydrogen atom and that when none of the radicals R′ and R″ represents a hydrogen atom, R′ and R″ are identical, or of a mixture of compounds of formula (I), said use being for the purpose of preventing or slowing down the appearance of wrinkles, fine lines, impairment of the microrelief, lack of elasticity and/or tonicity, lack of density and/or firmness of human skin or lips or alternatively for eliminating them and said use being in a cosmetic composition.

According to a first particular aspect, a subject of the invention is the use as defined above, of a composition (C1) comprising, per 100% of its mass:
from 99% by mass to 20% by mass, more particularly from 99% by mass to 50% by mass, and even more particularly from 95% by mass to 75%, of at least one compound of formula (Ia):

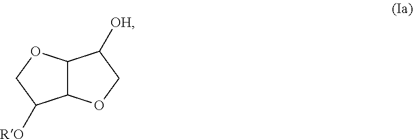

in which formula (Ia) R′ represents either a monovalent radical of formula (IIa):

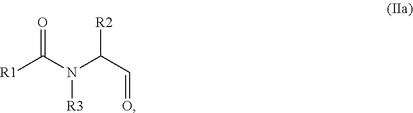

in which formula (IIa) the group R1-C(═O)— is chosen from octanoyl, decanoyl, ω-undecylenoyl, dodecanoyl, tetradecanoyl, hexadecanoyl, octadecanoyl, 9-octadecenoyl and 9,12-octadecadienoyl radicals, R3 represents a hydrogen atom and R2 represents a radical chosen from methyl, isopropyl, isobutyl and 1-methylpropyl radicals, and
from 1% by mass to 80% by mass, more particularly from 1% by mass to 50% by mass, and even more particularly from 5% by mass to 25% by mass, of at least one compound of formula (Ib):

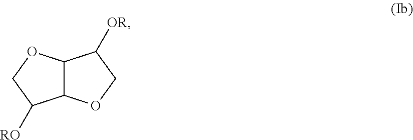

in which formula (Ib) R represents either a monovalent radical of formula (IIa):

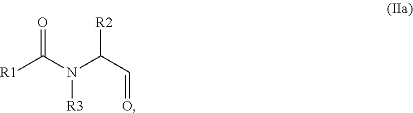

in which formula (IIa) the group R1-C(═O)— is chosen from octanoyl, decanoyl, ω-undecylenoyl, dodecanoyl, tetradecanoyl, hexadecanoyl, octadecanoyl, 9-octadecenoyl and 9,12-octadecadienoyl radicals, R3 represents a hydrogen atom and R2 represents a radical chosen from methyl, isopropyl, isobutyl and 1-methylpropyl radicals,

According to a particular aspect of the present invention, a subject thereof is the use as described previously of a compound of formula (I) or of a composition (C₁) as defined above for which the compound of formula (I), the compound of formula (Ia) and the compound of formula (Ib) are chosen from isosorbide esters of N-octanoyl alanine, of N-octanoyl valine, of N-octanoyl leucine, of N-octanoyl isoleucine, of N-(ω-undecylenoyl) alanine, of N-(ω-undecylenoyl) valine, of N-(ω-undecylenoyl) leucine, of N-(ω-undecylenoyl) isoleucine, of N-dodecanoyl alanine, of N-dodecanoyl valine, of N-dodecanoyl leucine, of N-dodecanoyl isoleucine, N-tetradecanoyl alanine, of N-tetradecanoyl valine, of N-tetradecanoyl leucine, of N-tetradecanoyl isoleucine, of N-hexadecanoyl alanine, of N-hexadecanoyl valine, of N-hexadecanoyl leucine, of N-hexadecanoyl isoleucine, of N-octadecanoyl alanine, of N-octadecanoyl valine, of N-octadecanoyl leucine, of N-octadecanoyl isoleucine, of N-oleoyl alanine, of N-oleoyl valine, of N-oleoyl leucine or of N-oleoyl isoleucine or for which the mixture of compounds of formula (I) is chosen from the isosorbide esters of N-cocoyl alanine, of N-cocoyl valine, of N-cocoyl leucine or of N-cocoyl isoleucine.

The terms N-cocoyl alanine, N-cocoyl valine, N-cocoyl leucine and N-cocoyl isoleucine denote the mixtures of N-acyl derivatives, respectively, of valine, of leucine or of isoleucine obtained by reaction of each of these α-amino acids with the mixture of fatty acid chloride derived from coconut oil comprising, per 100 mol %, about 11 mol % of octanoyl chloride, about 9.5 mol % of decanoyl chloride, about 51 mol % of lauroyl chloride, about 15.5 mol % of myristoyl chloride, about 6.5 mol % of palmitoyl chloride, about 2 mol % of stearoyl chloride, about 3 mol % of oleoyl chloride and about 1.5 mol % of linoleoyl chloride.

According to another more particular aspect of the present invention, a subject thereof is the use as described previously of a compound of formula (Ia) as defined previously, chosen from isosorbide N-octanoyl alaninate, isosorbide N-hexadecanoyl valinate, isosorbide N-octanoyl isoleucinate or the isosorbide N-cocoyl isoleucinate mixture.

According to another particular aspect of the present invention, a subject thereof is the use as described previously of a compound of formula (Ib) as defined previously, chosen from isosorbide bis(N-octanoyl alaninate), isosorbide bis(N-hexadecanoyl) valinate, isosorbide bis(N-octanoyl isoleucinate) or the isosorbide bis(N-cocoyl isoleucinate) mixture.

The compound of formula (I) as defined previously may be prepared according to a preparation process comprising:

a step a) of esterification either of a compound of formula (IIIa):

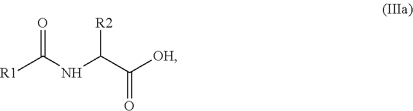

in which R1-C(═O)— and R2 are as defined in formula (IIa), or of a compound of formula (IIIb):

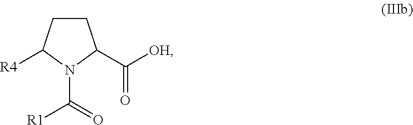

in which R1-C(═O)— and R4 are as defined in formula (IIb), with the isosorbide of formula (IV):

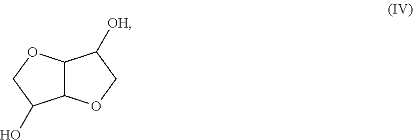

to obtain either the compound of formula (Ia) or the compound of formula (Ib), or a mixture (M) of the compound of formula (Ia) and of the compound of formula (Ib); and, if necessary or if desired,

a step b) of separation of the compounds of formula (Ia) and of formula (Ib), from said mixture (M) obtained in step (a).

The compounds of formulae (IIIa) and (IIIb) are known or are synthesizable by N-acylation of the corresponding α-amino acids according to methods known to those skilled in the art.

In the process as defined above, the mole ratio of the compound of formula (IIIa) or of formula (IIIb) to the isosorbide of formula (IV) is generally between 3/1 and 1/5, more particularly between 1/1 and 1/5 and even more particularly between 1/1 and 1/3.

In the process as defined above, step b) of separation of the compounds of formula (Ia) and of formula (Ib) is performed via the standard separation methods known to those skilled in the art.

The compound of formula (I) as defined previously may also be prepared according to a preparation process comprising:

a step a1) of esterification either of a compound of formula (IIIa):

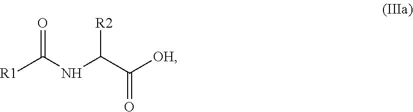

in which R1 and R2 are as defined in formula (IIa), or of a compound of formula (IIIb):

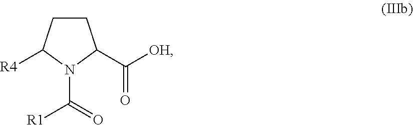

in which R1 and R4 are as defined for formula (IIb), with an alcohol of formula (V):

R5-OH (V),

in which R5 represents a linear aliphatic radical comprising from 1 to 4 carbon atoms, to form either a compound of formula (VIa):

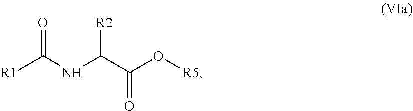

in which R1, R2 and R5 are as defined previously, or of a compound of formula (VIb):

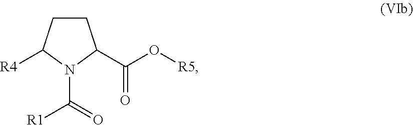

in which R1, R4 and R5 are as defined previously;

a step a2) of trans-esterification of the compound of formula (VIa) or of the compound of formula (VIb) obtained in step a1), by reaction with the isosorbide of formula (IV), to obtain either the compound of formula (Ia) or the compound of formula (Ib), or a mixture (M) of the compound of formula (Ia) and of the compound of formula (Ib); and, if necessary or if desired,

the implementation of step b).

In the process as described above, step a1) is generally performed at a temperature of about between 60° C. and 120° C. under inert gas, and in the presence of an acidic catalytic system. The term “acidic catalytic system” denotes strong acids such as sulfuric acid, hydrochloric acid, phosphoric acid, nitric acid, hypophosphorous acid, methanesulfonic acid, para-toluenesulfonic acid, trifluoromethanesulfonic acid, or acidic ion-exchange resins.

In step a1) of the process as described above, the mole ratio of the compound of formula (IIIa) or the compound of formula (IIIb) to the alcohol of formula (V) is generally between 1/1 and 1/10, more particularly between 1/1 and 1/8 and even more particularly between 1/2 and 1/8.

In the process as described above, step a2) of transesterification of the ester of formula (VIa) or the compound of formula (VIb) obtained in step a1) is generally performed at a temperature of about between 80° C. and 180° C., more particularly between 100° C. and 150° C. and even more particularly between 120° C. and 150° C., under an inert gas, and in the presence of an acidic catalytic system as described previously, and with distillation under vacuum of the alcohol of formula (V) formed in situ.

In step a2) of the process as described above, the mole ratio of the compound of formula (V) to the isosorbide of formula (V) is between 3/1 and 1/5, more particularly between 1/1 and 1/5 and even more particularly between 1/1 and 1/3.

Composition (C1) as defined previously may be prepared via various routes.

A first route for preparing composition (C1), performed in the use that is the subject matter of the invention, consists in mixing, in the desired mass proportions, the compound of formula (Ia) as defined above with the compound of formula (Ib) as defined above.

A second route for preparing composition (C1), performed in the use that is the subject matter of the invention, consists in performing the process for preparing the compound of formula (I) as described previously, by reacting, in the desired proportions, the isosorbide of formula (IV) with the compound of formula (IIIa) and/or this compound of formula (IIIb).

A third route for preparing composition (C1), performed in the use that is the subject matter of the invention, consists in performing the variant of the process for preparing the compound of formula (I) as described previously, by reacting, in the desired proportions, the compound of formula (IIIa) or compound (IIIb) with the alcohol of formula (V), and then the isosorbide of formula (IV).

A subject of the invention is also a process for the purpose of preventing or slowing the appearance of wrinkles or fine lines, impairment of the microrelief, lack of elasticity and/or tonicity, lack of density and/or firmness or else for eliminating them, comprising at least one step of applying to human skin or to the lips a cosmetic formulation for topical use comprising at least one cosmetically acceptable excipient and an effective amount of at least one compound of formula (I) or of a composition (C1) as defined previously.

In the cosmetic process as described above, the cosmetic formulation for topical use is spread onto the surface of the skin to be treated, and the skin is then massaged for a few moments.

The expression “for topical use” used in the definition of the cosmetic formulation used in the cosmetic process that is the subject of the present invention means that said formulation is used by application to the skin, whether it is direct application in the case of a cosmetic formulation or indirect application when the cosmetic formulation according to the invention is impregnated on a support intended to be placed in contact with the skin (paper, wipe, textile, transdermal device, etc.).

The expression “cosmetically acceptable” used in the definition of the cosmetic formulation for topical use, used in the cosmetic process that is the subject of the present invention, means, according to the European Economic Community Council directive No. 76/768/EEC of Jul. 27, 1976 amended by directive No. 93/35/EEC of Jun. 14, 1993, that said formulation comprises any substance or preparation intended to be placed in contact with the various parts of the human body (epidermis, pilous and hair system, nails, lips and genital organs) or with the teeth and the oral mucosae for the purpose, exclusively and mainly, of cleansing them, fragrancing them, modifying their appearance and/or correcting their body odor and/or protecting them or keeping them in good condition.

The term “effective amount of the compound of formula (I) or of composition (C1) as defined previously present in the cosmetic formulation for topical use used in the process as defined above” means, per 100% of the mass of said cosmetic formulation for topical use, the amount between 0.1% and 5% by mass, more particularly between 0.1% and 3% by mass and even more particularly between 0.5% and 2% by mass of compound of formula (I) or of composition (C1).

The cosmetic formulations for topical use used in the cosmetic process as defined above are generally in the form of aqueous or aqueous-alcoholic or water-glycol solutions, in the form of a suspension, an emulsion, a micro-emulsion or a nano-emulsion, whether they are of the water-in-oil, oil-in-water, water-in-oil-in-water or oil-in-water-in-oil type, or in the form of a powder.

The cosmetic formulations for topical use used in the cosmetic process that is the subject of the present invention may be packaged in a bottle, in a device of pump-bottle type, in pressurized form in an aerosol device, in a device equipped with an openwork wall such as a grate or in a device equipped with a ball applicator (roll-on).

In general, the compound of formula (I) or composition (C1) present in the cosmetic formulations for topical use used in the cosmetic process that is the subject of the present invention is combined with chemical additives usually used in the field of formulations for topical use, such as foaming and/or detergent surfactants, thickening and/or gelling surfactants, thickening and/or gelling agents, stabilizers, film-forming compounds, solvents and co-solvents, hydrotropic agents, spring or mineral waters, plasticizers, emulsifiers and co-emulsifiers, opacifiers, nacreous agents, overfatting agents, sequestrants, chelating agents, oils, waxes, antioxidants, fragrances, essential oils, preserving agents, conditioning agents, deodorizers, bleaching agents intended for bleaching skin and bodily hairs, active principles intended for providing a treating and/or protective action on the skin or hair, sunscreens, mineral fillers or pigments, particles affording a visual effect or intended for encapsulating active agents, exfoliant particles, texturing agents, optical brighteners and insect repellents.

As examples of foaming and/or detergent surfactants that may be combined with the compound of formula (I) or with composition (C1) in the cosmetic formulations for topical use used in the cosmetic process that is the subject of the present invention, mention may be made of anionic, cationic, amphoteric or nonionic foaming and/or detergent surfactants.

Among the foaming and/or detergent anionic surfactants that may be combined with the compound of formula (I) or composition (C1) in the cosmetic formulations for topical use used in the cosmetic process that is the subject of the present invention, mention may be made of salts of alkali metals, of alkaline-earth metals, of ammonium, of amines, or of amino alcohols, of alkyl ether sulfates, of alkyl sulfates, of alkylamido ether sulfates, of alkylaryl polyether sulfates, of monoglyceride sulfates, of alpha-olefin sulfonates, of paraffin sulfonates, of alkyl phosphates, of alkyl ether phosphates, of alkylsulfonates, of alkylamide sulfonates, of alkylarylsulfonates, of alkylsulfosuccinates, of alkyl ether sulfosuccinates, of alkylamide sulfosuccinates, of alkylsulfoacetates, of alkylsarcosinates, of acylisethionates, of N-acyltaurates, of acyllactylates, of N-acylamino acid derivatives, of N-acyl peptide derivatives, of N-acyl protein derivatives or of N-acyl fatty acid derivatives.

Among the foaming and/or detergent amphoteric surfactants that may be combined with the compound of formula (I) or with composition (C1) in the cosmetic formulations for topical use used in the cosmetic process that is the subject of the present invention, mention may be made of alkylbetaines, alkylamidobetaines, sultaines, alkylamidoalkylsulfobetaines, imidazoline derivatives, phosphobetaines, amphopolyacetates and amphopropionates.

As examples of foaming and/or detergent cationic surfactants that may be combined with the compound of formula (I) or with composition (C1) in the cosmetic formulations for topical use used in the cosmetic process that is the subject of the present invention, mention may be made particularly of quaternary ammonium derivatives.

Among the foaming and/or detergent nonionic surfactants that may be combined with the compound of formula (I) or with composition (C1) in the cosmetic formulations for topical use used in the cosmetic process that is the subject of the present invention, mention may be made more particularly of alkylpolyglycosides bearing a linear or branched, saturated or unsaturated aliphatic radical and comprising from 8 to 16 carbon atoms, such as octyl polyglucoside, decyl polyglucoside, undecylenyl polyglucoside, dodecyl polyglucoside, tetradecyl polyglucoside, hexadecyl polyglucoside, 1,12-dodecanediyl polyglucoside; ethoxylated hydrogenated castor oil derivatives, such as the product sold under the INCI name PEG-40 hydrogenated castor oil; polysorbates such as Polysorbate 20, Polysorbate 40, Polysorbate 60, Polysorbate 70, Polysorbate 80 or Polysorbate 85; coconut amides; N-alkylamines.

As examples of thickening and/or gelling surfactants that may be combined with the compound of formula (I) or with composition (C1) in the cosmetic formulations for topical use used in the cosmetic process that is the subject of the present invention, mention may be made of optionally poly(alkoxylated) alkylpolyglycoside fatty esters, for instance ethoxylated methylpolyglucoside esters such as PEG 120 methyl glucose trioleate and PEG 120 methyl glucose dioleate sold, respectively, under the names Glucamate™ LT and Glumate™ DOE120; alkoxylated fatty esters such as PEG 150 pentaerythrityl tetrastearate sold under the name Crothix™ DS53, or PEG 55 propylene glycol oleate sold under the name Antil™ 141; fatty-chain polyalkylene glycol carbamates such as PPG-14 laureth isophoryl dicarbamate sold under the name Elfacos™ T211, PPG-14 palmeth-60 hexyl dicarbamate sold under the name Elfacos™ GT2125.

As examples of thickeners and/or gelling agents that may be combined with the compound of formula (I) or with composition (C1) in the cosmetic formulations for topical use used in the cosmetic process that is the subject of the present invention, mention may be made of polymers such as linear or branched or crosslinked polyelectrolytes, for instance the partially or totally salified acrylic acid homopolymer, the partially or totally salified methacrylic acid homopolymer, the partially or totally salified 2-methyl[(1-oxo-2-propenyl)amino]-1-propanesulfonic acid (AMPS) homopolymer, copolymers of acrylic acid and of AMPS, copolymers of acrylamide and of AMPS, copolymers of vinylpyrrolidone and of AMPS, copolymers of AMPS and of (2-hydroxyethyl) acrylate, copolymers of AMPS and of (2-hydroxyethyl) methacrylate, copolymers of AMPS and of hydroxyethylacrylamide, copolymers of AMPS and of N,N-dimethylacrylamide, copolymers of AMPS and of tris(hydroxymethyl)acrylamidomethane (THAM), copolymers of acrylic or methacrylic acid and of (2-hydroxyethyl) acrylate, copolymers of acrylic or methacrylic acid and of (2-hydroxyethyl) methacrylate, copolymers of acrylic or methacrylic acid and of hydroxyethylacrylamide, copolymers of acrylic or methacrylic acid and of THAM, copolymers of acrylic or methacrylic acid and of N,N-dimethylacrylamide, terpolymers of acrylic or methacrylic acid, of AMPS and of (2-hydroxyethyl) acrylate, terpolymers of acrylic or methacrylic acid, of AMPS and of (2-hydroxyethyl) methacrylate, terpolymers of acrylic or methacrylic acid, of AMPS and of THAM, terpolymers of acrylic or methacrylic acid, of AMPS and of N,N-dimethylacrylamide, terpolymers of acrylic or methacrylic acid, of AMPS and of acrylamide, copolymers of acrylic acid or of methacrylic acid and of alkyl acrylates in which the carbon-based chain comprises between 4 and 30 carbon atoms and more particularly between 10 and 30 carbon atoms, copolymers of AMPS and of alkyl acrylates in which the carbon-based chain comprises between 4 and 30 carbon atoms and more particularly between 10 and 30 carbon atoms, linear, branched or crosslinked terpolymers of at least one monomer bearing a free, partially salified or totally salified strong acid function, with at least one neutral monomer, and at least one monomer of formula (VIII):

CH₂═C(R6)-C(═O)—[CH₂—CH₂—O]ₙ—R7 (VIII)

in which R6 represents a hydrogen atom or a methyl radical, R7 represents a linear or branched alkyl radical comprising from 8 to 30 carbon atoms and n represents a number greater than or equal to 1 and less than or equal to 50.

The polymers such as linear or branched or crosslinked polyelectrolytes that may be combined with the compound of formula (I) or composition (C1) in the cosmetic formulations for topical use used in the cosmetic process that is the subject of the present invention may be in the form of a solution, an aqueous suspension, a water-in-oil emulsion, an oil-in-water emulsion or a powder. The polymers such as linear or branched or crosslinked polyelectrolytes that may be combined with the compound of formula (I) or with composition (C1) in the cosmetic formulations for topical use used in the cosmetic process that is the subject of the present invention may be selected from the products sold under the names Simulgel™ EG, Simulgel™ EPG, Sepigel™ 305, Simulgel™ 600, Simulgel™ NS, Simulgel™ INS 100, Simulgel™ FL, Simulgel™ A, Simulgel™ SMS 88, Sepinov™ EMT 10, Sepiplus™ 400, Sepiplus™ 265, Sepiplus™ S, Sepimax™ Zen, Aristoflex™ AVC, Aristoflex™ AVS, Novemer™ EC-1, Novemer™ EC 2, Aristoflex™ HMB, Cosmedia™ SP, Flocare™ ET 25, Flocare™ ET 75, Flocare™ ET 26, Flocare™ ET 30, Flocare™ ET 58, Flocare™ PSD 30, Viscolam™ AT 64 and Viscolam™ AT 100.

As examples of thickeners and/or gelling agents that may be combined with the compound of formula (I) or with composition (C1) in the cosmetic formulations for topical use used in the cosmetic process that is the subject of the present invention, mention may be made of polysaccharides constituted solely of saccharides, for instance glucans or glucose homopolymers, glucomannoglucans, xyloglycans, galactomannans in which the degree of substitution (DS) of the D-galactose units on the main D-mannose chain is between 0 and 1, and more particularly between 1 and 0.25, such as galactomannans originating from cassia gum (DS=1/5), from locust bean gum (DS=1/4), from tara gum (DS=1/3), from guar gum (DS=1/2) and from fenugreek gum (DS=1).

As examples of thickeners and/or gelling agents that may be combined with the compound of formula (I) or with composition (C1) in the cosmetic formulations for topical use used in the cosmetic process that is the subject of the present invention, mention may be made of polysaccharides constituted of saccharide derivatives, such as galactan sulfates and more particularly carrageenans and agar, uronans and more particularly algins, alginates and pectins, heteropolymers of saccharides and of uronic acids and more particularly xanthan gum, gellan gum, gum arabic and karaya gum exudates, and glucosaminoglycans.

As examples of thickeners and/or gelling agents that may be combined with the compound of formula (I) or with composition (C1) in the cosmetic formulations for topical use used in the cosmetic process that is the subject of the present invention, mention may be made of cellulose, cellulose derivatives such as methylcellulose, ethylcellulose, hydroxypropylcellulose, silicates, starch, hydrophilic starch derivatives and polyurethanes.

As examples of stabilizers that may be combined with the compound of formula (I) or with composition (C1) in the cosmetic formulations for topical use used in the cosmetic process that is the subject of the present invention, mention may be made of microcrystalline waxes, and more particularly ozokerite, mineral salts such as sodium chloride or magnesium chloride, and silicone polymers such as polysiloxane polyalkyl polyether copolymers.

As examples of solvents that may be combined with the compound of formula (I) or with composition (C1) in the cosmetic formulations for topical use used in the cosmetic process that is the subject of the present invention, mention may be made of water, organic solvents such as glycerol, diglycerol, glycerol oligomers, ethylene glycol, propylene glycol, butylene glycol, 1,3-propanediol, 1,2-propanediol, hexylene glycol, diethylene glycol, xylitol, erythritol, sorbitol, water-soluble alcohols such as ethanol, isopropanol or butanol, and mixtures of water and of said organic solvents.

As examples of spring or mineral waters that may be combined with the compound of formula (I) or with composition (C1) in the cosmetic formulations for topical use used in the cosmetic process that is the subject of the present invention, mention may be made of spring or mineral waters with a mineralization of at least 300 mg/I, in particular Avène water, Vittel water, Vichy basin water, Uriage water, La Roche-Posay water, La Bourboule water, Enghien-les-Bains water, Saint Gervais-les-Bains water, Néris-les-Bains water, Allevard-les-Bains water, Digne water, Maizières water, Neyrac-les-Bains water, Lons-le-Saunier water, Rochefort water, Saint Christau water, Les Fumades water and Tercis-les-Bains water.

As examples of hydrotropic agents that may be combined with the compound of formula (I) or with composition (C1) in the cosmetic formulations for topical use used in the cosmetic process that is the subject of the present invention, mention may be made of xylene sulfonates, cumene sulfonates, hexyl polyglucoside, (2-ethylhexyl) polyglucoside and n-heptyl polyglucoside.

As examples of emulsifying surfactants that may be combined with the compound of formula (I) or with composition (C1) in the cosmetic formulations for topical use used in the cosmetic process that is the subject of the present invention, mention may be made of nonionic surfactants, anionic surfactants and cationic surfactants.

As examples of emulsifying nonionic surfactants that may be combined with the compound of formula (I) or with composition (C1) in the cosmetic formulations for topical use used in the cosmetic process that is the subject of the present invention, mention may be made of fatty acid esters of sorbitol, for example the products sold under the names Montane™ 40 and Montane™ 60, Montane™ 70, Montane™ 80 and Montane™ 85; compositions comprising glyceryl stearate and stearic acid ethoxylated between 5 mol and 150 mol of ethylene oxide, for example the composition comprising stearic acid ethoxylated with 135 mol of ethylene oxide and glyceryl stearate sold under the name Simulsol™ 165; mannitan esters; ethoxylated mannitan esters; sucrose esters; methylglucoside esters; alkylpolyglycosides comprising a linear or branched, saturated or unsaturated aliphatic radical, and comprising from 14 to 36 carbon atoms, such as tetradecyl polyglucoside, hexadecyl polyglucoside, octadecyl polyglucoside, hexadecyl polyxyloside, octadecyl polyxyloside, eicosyl polyglucoside, dodecosyl polyglucoside, 2-octyldodecyl polyxyloside, 12-hydroxystearyl polyglucoside; compositions of linear or branched, saturated or unsaturated fatty alcohols, comprising from 14 to 36 carbon atoms, and of alkyl polyglucosides as described previously.

As examples of anionic surfactants that may be combined with the compound of formula (I) or with composition (C1) in the cosmetic formulations for topical use used in the cosmetic process that is the subject of the present invention, mention may be made of glyceryl stearate citrate, cetearyl sulfate, soaps such as sodium stearate or triethanolammonium stearate, salified N-acylamino acid derivatives, for example stearoyl glutamate.

As examples of emulsifying cationic surfactants that may be combined with the compound of formula (I) or with composition (C1) in the cosmetic formulations for topical use used in the cosmetic process that is the subject of the present invention, mention may be made of aminoxides, quaternium-82 and the surfactants described in patent application WO 96/00719 and mainly those whose fatty chain comprises at least 16 carbon atoms.

As examples of opacifiers and/or nacreous agents that may be combined with the compound of formula (I) or with composition (C1) in the cosmetic formulations for topical use used in the cosmetic process that is the subject of the present invention, mention may be made of sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate, and fatty alcohols comprising from 12 to 22 carbon atoms.

As examples of texturing agents that may be combined with the compound of formula (I) or with composition (C1) in the cosmetic formulations for topical use used in the cosmetic process that is the subject of the present invention, mention may be made of N-acylamino acid derivatives, such as lauroyl lysine sold under the name Aminohope™ LL, octenyl starch succinate sold under the name DryFlo™, myristyl polyglucoside sold under the name Montanov™ 14, cellulose fibers, cotton fibers, chitosan fibers, talc, sericite and mica.

As examples of deodorants that may be combined with the compound of formula (I) or with composition (C1) in the cosmetic formulations for topical use used in the cosmetic process that is the subject of the present invention, mention may be made of alkaline silicates, zinc salts such as zinc sulfate, zinc gluconate, zinc chloride or zinc lactate; quaternary ammonium salts such as cetyltrimethylammonium salts, and cetylpyridinium salts; glycerol derivatives such as glyceryl caprate, glyceryl caprylate, polyglyceryl caprate; 1,2-decanediol, 1,3-propanediol; salicylic acid; sodium bicarbonate; cyclodextrins; metal zeolites; Triclosan™; aluminum hydrobromide, aluminum hydrochlorides, aluminum chloride, aluminum sulfate, aluminum zirconium hydrochlorides, aluminum zirconium trihydrochloride, aluminum zirconium tetrahydrochloride, aluminum zirconium pentahydrochloride, aluminum zirconium octahydrochloride, aluminum sulfate, sodium aluminum lactate, complexes of aluminum hydrochloride and glycol, such as the complex of aluminum hydrochloride and of propylene glycol, the complex of aluminum dihydrochloride and of propylene glycol, the complex of aluminum sesquihydrochloride and of propylene glycol, the complex of aluminum hydrochloride and of polyethylene glycol, the complex of aluminum dihydrochloride and of polyethylene glycol, the complex of aluminum sesquihydrochloride and of polyethylene glycol.

As examples of oils that may be combined with the compound of formula (I) or with composition (C1) in the cosmetic formulations for topical use used in the cosmetic process that is the subject of the present invention, mention may be made of mineral oils such as liquid paraffin, liquid petroleum jelly, isoparaffins or white mineral oils; oils of animal origin, such as squalene or squalane; plant oils, such as phytosqualane, sweet almond oil, coconut oil, castor oil, jojoba oil, olive oil, rapeseed oil, groundnut oil, sunflower oil, wheatgerm oil, corn germ oil, soybean oil, cotton oil, alfalfa oil, poppy oil, pumpkin oil, evening primrose oil, millet oil, barley oil, rye oil, safflower oil, candlenut oil, passion flower oil, hazelnut oil, palm oil, shea butter, apricot kernel oil, beauty-leaf oil, sysymbrium oil, avocado oil, calendula oil, oils derived from flowers or vegetables, ethoxylated plant oils; synthetic oils, for instance fatty acid esters such as butyl myristate, propyl myristate, isopropyl myristate, cetyl myristate, isopropyl palmitate, octyl palmitate, butyl stearate, hexadecyl stearate, isopropyl stearate, octyl stearate, isocetyl stearate, dodecyl oleate, hexyl laurate, propylene glycol dicaprylate, lanolic acid-based esters, such as isopropyl lanolate, isocetyl lanolate, fatty acid monoglycerides, diglycerides and triglycerides, for instance glyceryl triheptanoate, alkylbenzoates, hydrogenated oils, poly(alpha-olefins), polyolefins such as poly(isobutane), synthetic isoalkanes such as isohexadecane, isododecane, perfluoro oils; silicone oils such as dimethylpolysiloxanes, methylphenylpolysiloxanes, silicones modified with amines, silicones modified with fatty acids, silicones modified with alcohols, silicones modified with alcohols and fatty acids, silicones modified with polyether groups, modified epoxy silicones, silicones modified with fluoro groups, cyclic silicones and silicones modified with alkyl groups. In the present patent application, the term “oils” means compounds and/or mixtures of compounds that are insoluble in water, which have a liquid appearance at a temperature of 25° C.

As examples of waxes that may be combined with the compound of formula (I) or with composition (C1) in the cosmetic formulations for topical use used in the cosmetic process that is the subject of the present invention, mention may be made of beeswax, carnauba wax, candelilla wax, ouricury wax, Japan wax, cork fiber wax, sugar cane wax, paraffin waxes, lignite waxes, microcrystalline waxes, lanolin wax; ozokerite; polyethylene wax; silicone waxes; plant waxes; fatty alcohols and fatty acids that are solid at room temperature; glycerides that are solid at room temperature. In the present patent application, the term “waxes” means water-insoluble compounds and/or mixtures of compounds, which have a solid appearance at a temperature of greater than or equal to 45° C.

As examples of active principles that may be combined with the compound of formula (I) or with composition (C1) in the cosmetic formulations for topical use used in the cosmetic process that is the subject of the present invention, mention may be made of vitamins and derivatives thereof, especially esters thereof, such as retinol (vitamin A) and esters thereof (for example retinyl palmitate), ascorbic acid (vitamin C) and esters thereof, sugar derivatives of ascorbic acid (such as ascorbyl glucoside), tocopherol (vitamin E) and esters thereof (such as tocopheryl acetate), vitamin B3 or B10 (niacinamide and derivatives thereof); compounds which show lightening or depigmenting action on the skin, such as ω-undecelynoyl phenylalanine sold under the name Sepiwhite™ MSH, Sepicalm™ VG, the glyceryl monoester and/or diester of ω-undecelynoyl phenylalanine, ω-undecelynoyl dipeptides, arbutin, kojic acid, hydroquinone; compounds showing calmative action, especially Sepicalm™ S, allantoin and bisabolol; antiinflammatory agents; compounds showing moisturizing action, such as urea, hydroxyureas, glycerol, polyglycerols, glycerol glucoside, diglycerol glucoside, polyglyceryl glucosides, xylityl glucoside; polyphenol-rich plant extracts such as grape extracts, pine extracts, vine extracts and olive extracts; compounds showing slimming or lipolytic action such as caffeine or derivatives thereof, Adiposlim™, Adipoless™ fucoxanthin; N-acyl proteins; N-acyl peptides such as Matrixyl™; N-acylamino acids; N-acyl partial protein hydrolysates; amino acids; peptides; total protein hydrolyzates; soybean extracts, for example Raffermine™; wheat extracts, for example Tensine™ or Gliadine™ plant extracts, such as tannin-rich plant extracts, isoflavone-rich plant extracts or terpene-rich plant extracts; freshwater or marine water algal extracts; marine plant extracts; marine extracts in general such as corals; essential waxes; bacterial extracts; ceramides; phospholipids; compounds showing antimicrobial action or purifying action, such as Lipacide™ C8G, Lipacide™ UG, Sepicontrol™ A5; Octopirox™ or Sensiva™ SC50; compounds showing an energizing or stimulating property such as Physiogenyl™, panthenol and derivatives thereof such as Sepicap™ MP; antiaging active agents such as Sepilift™ DPHP, Lipacide™ PVB, Sepivinol™ Sepivital™ Manoliva™ Phyto-Age™, Timecode™; Survicode™; anti-photoaging active agents; active agents for protecting the integrity of the dermo-epidermal junction; active agents for increasing the synthesis of components of the extracellular matrix, such as collagen, elastins and glycosaminoglycans; active agents acting favorably on chemical cellular communication, such as cytokines, or physical cellular communication, such as integrins; active agents creating a sensation of “heating” on the skin, such as skin microcirculation activators (such as nicotinic acid derivatives) or products that create a sensation of “freshness” on the skin (such as menthol and derivatives thereof); active agents which improve the skin microcirculation, for example venotonic agents; draining active agents; decongestant active agents such as Ginkgo biloba, ivy, common horse chestnut, bamboo, ruscus, butcher's-broom, Centella asiatica, fucus, rosemary or willow extracts; skin tanning or browning agents, for instance dihydroxyacetone, isatin, alloxan, ninhydrin, glyceraldehyde, mesotartaric aldehyde, glutaraldehyde or erythrulose.

As examples of antioxidants that may be combined with the compound of formula (I) or with composition (C1) in the cosmetic compositions for topical use used in the cosmetic process that is the subject of the present invention, mention may be made of EDTA and salts thereof, citric acid, tartaric acid, oxalic acid, BHA (butyl hydroxyanisole), BHT (butyl hydroxytoluene), tocopherol derivatives such as tocopheryl acetate, mixtures of antioxidant compounds such as Dissolvine™ GL 47S sold by the company Akzo Nobel under the INCI name: Tetrasodium glutamate diacetate.

As examples of sunscreens that may be combined with the compound of formula (I) or with composition (C1) in the cosmetic formulations for topical use used in the cosmetic process that is the subject of the present invention, mention may be made of all those featured in the cosmetic directive 76/768/EEC amended annex VII.

Among the organic sunscreens that may be combined with the compound of formula (I) or with composition (C1) in the cosmetic formulations for topical use used in the cosmetic process that is the subject of the present invention, mention may be made of the family of benzoic acid derivatives for instance para-aminobenzoic acids (PABA), especially monoglyceryl esters of PABA, ethyl esters of N,N-propoxy PABA, ethyl esters of N,N-diethoxy PABA, ethyl esters of N,N-dimethyl PABA, methyl esters of N,N-dimethyl PABA and butyl esters of N,N-dimethyl PABA; the family of anthranilic acid derivatives, for instance homomenthyl-N-acetyl anthranilate; the family of salicylic acid derivatives, for instance amyl salicylate, homomenthyl salicylate, ethylhexyl salicylate, phenyl salicylate, benzyl salicylate and p-isopropanolphenyl salicylate; the family of cinnamic acid derivatives, for instance ethylhexyl cinnamate, ethyl-4-isopropyl cinnamate, methyl-2,5-diisopropyl cinnamate, p-methoxypropyl cinnamate, p-methoxyisopropyl cinnamate, p-methoxyisoamyl cinnamate, p-methoxyoctyl cinnamate (p-methoxy 2-ethylhexyl cinnamate), p-methoxy-2-ethoxyethyl cinnamate, p-methoxycyclohexyl cinnamate, ethyl-α-cyano-β-phenyl cinnamate, 2-ethylhexyl-α-cyano-β-phenyl cinnamate or glyceryl di-para-methoxy-mono-2-ethylhexanoyl cinnamate; the family of benzophenone derivatives, for instance 2,4-dihydroxybenzophenone, 2,2′-dihydroxy-4-methoxybenzophenone, 2,2′,4,4′-tetrahydroxybenzophenone, 2-hydroxy-4-m ethoxybenzophenone, 2-hydroxy-4-m ethoxy-4′-m ethylbenzophenone, 2-hydroxy-4-methoxybenzophenone-5-sulfonate, 4-phenylbenzophenone, 2-ethylhexyl-4′-phenylbenzophenone-2-carboxylate, 2-hydroxy-4-n-octyloxybenzophenone, 4-hydroxy-3-carboxybenzophenone; 3-(4′-methylbenzylidene)-d,l-camphor, 3-(benzylidene)-d,l-camphor, camphor benzalkonium methosulfate; urocanic acid, ethyl urocanate; the family of sulfonic acid derivatives, for instance 2-phenylbenzimidazole-5-sulfonic acid and salts thereof; the family of triazine derivatives, for instance hydroxyphenyltriazine, (ethylhexyloxyhydroxyphenyl)(4-methoxyphenyl)triazine, 2,4,6-trianillino(p-carbo-2′-ethylhexyl-1′-oxy)-1,3,5-triazine, 4,4-((6-(((1,1-dimethylethyl)amino)carbonyl)phenyl)amino)-1,3,5-triazine-2,4-diyldiimino)bis-(2-ethylhexyl) benzoate, 2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methylphenyl)benzotriazole; dibenzazine; dianisoylmethane, 4-methoxy-4″-t-butylbenzoylmethane; 5-(3,3-dimethyl-2-norbornylidene)-3-pentan-2-one; the family of diphenylacrylate derivatives, for instance 2-ethylhexyl-2-cyano-3,3-diphenyl-2-propenoate, ethyl-2-cyano-3,3-diphenyl-2-propenoate; the family of polysiloxanes, for instance benzylidene siloxane malonate.

Among the mineral sunscreens, also known as “mineral sunblocks”, which may be combined with the compound of formula (I) or with composition (C1) in the cosmetic formulations for topical use used in the cosmetic process that is the subject of the present invention, mention may be made of titanium oxides, zinc oxides, cerium oxide, zirconium oxide, yellow, red or black iron oxides, and chromium oxides. These mineral sunblocks may or may not be micronized, may or may not have undergone surface treatments and may be optionally in the form of aqueous or oily predispersions.

The examples that follow illustrate the invention without, however, limiting it.

### EXAMPLE 1: PREPARATION OF A COMPOSITION (C1_{A}) ACCORDING TO THE INVENTION

500 g of alanine, i.e. 1 molar equivalent, are introduced into an aqueous-alcoholic mixture consisting of 1800 g of water and 200 g of isopropanol at a temperature of 20° C. The pH of the medium is adjusted to 10 by adding 30% sodium hydroxide solution. 731.7 g of octanoyl chloride, i.e. 0.8 molar equivalent, are then gradually added to the mixture maintained between 20° C. and 50° C. and at a pH of between 10 and 10.5.

The reaction medium is then kept stirring for 2 hours and then heated to reach 70° C., followed by addition of 979.6 g of an acidic solution of 75% phosphoric acid to gradually reach a pH value equal to 2.0. The aqueous phase of the medium is separated out by settling and the organic phase remaining in the reactor is washed several times with brine at room temperature with stirring. On conclusion of washing and then drying by distillation under vacuum of the residual water, the organic phase comprising 922.3 g of the desired N-octanoyl alanine is obtained.

133.8 g of isosorbide, i.e. 1 molar equivalent, are introduced with stirring into the reactor comprising a fraction of the preceding organic phase, which contains 0.8 molar equivalent of N-octanoyl alanine. The temperature is brought to 120° C., 0.73 g of 98% sulfuric acid and 0.73 g of 50% hypophosphorous acid are then added and the resulting mixture is brought to 125° C. under partial vacuum while sparging with nitrogen. The reaction mixture is then kept stirring for 10 hours at this temperature, and then neutralized by adding 30% sodium hydroxide solution so as to obtain a pH of composition (C1_{A}), diluted to 5% in water, of between 3.0 and 6.0.

The analytical characteristics of composition (C1_{A}) obtained are as follows:

Acid number (according to method NFT 60-204)=60.3
pH 5% of composition A in water (according to method NFT 73-206)=3.7
Hydroxyl number (according to U.S. Pharmacopea XXI NF XVI 01/011985)=261.9
Ester number (calculated by the difference between the saponification number measured according to method NFT 60-110 and the acid number measured according to method NFT 60-204)=104.4
Saponification number (according to NFT 60-206)=164.7 mg KOH/g

### EXAMPLE 2: PREPARATION OF A COMPOSITION (C1_{B}) ACCORDING TO THE INVENTION

The procedure of the process described in Example 1 is performed, replacing the molar equivalent of alanine with a molar equivalent of valine and the 0.8 molar equivalent of octanoyl chloride with 0.8 molar equivalent of (ω-undecylenoyl) chloride to obtain composition (C1_{B}), the analytical characteristics of which are as follows:

Acid number (according to NFT 60-204)=35.4
pH 5% of composition (C1_{B}) in water (according to method NFT 73-206)=5.0
Hydroxyl number (according to U.S. Pharmacopea XXI NF XVI 01/011985)=155.8
Ester number (calculated by the difference between the saponification number measured according to method NFT 60-110 and the acid number measured according to method NFT 60-204)=91.3
Saponification number (according to NFT 60-206)=126.7 mg KOH/g

### EXAMPLE 3: PREPARATION OF A COMPOSITION (C1_{C}) ACCORDING TO THE INVENTION

The procedure of the process described in Example 1 is performed, replacing the molar equivalent of alanine with a molar equivalent of valine and the 0.8 molar equivalent of octanoyl chloride with 0.8 molar equivalent of hexadecanoyl chloride to obtain composition (C1_{C}), the analytical characteristics of which are as follows:

Acid number (according to NFT 60-204)=12.3
pH 5% of composition (C1_{C}) in water (according to method NFT 73-206)=7.5
Hydroxyl number (according to U.S. Pharmacopea XXI NF XVI 01/011985)=100.2
Ester number (calculated by the difference between the saponification number measured according to method NFT 60-110 and the acid number measured according to method NFT 60-204)=96.2
Saponification number (according to NFT 60-206)=108.5 mg KOH/g

### EXAMPLE 4: PREPARATION OF A COMPOSITION (C1_{D}) ACCORDING TO THE INVENTION

The procedure of the process described in Example 1 is performed, replacing the molar equivalent of alanine with a molar equivalent of isoleucine, to obtain composition (C1_{D}), the analytical characteristics of which are as follows:

Acid number (according to NFT 60-204)=71.5
pH 5% of composition E in water (according to method NFT 73-206)=4.1
Hydroxyl number (according to U.S. Pharmacopea XXI NF XVI 01/011985)=219.8
Ester number (calculated by the difference between the saponification number measured according to method NFT 60-110 and the acid number measured according to method NFT 60-204)=86.2
Saponification number (according to NFT 60-206)=157.7 mg KOH/g

### EXAMPLE 5: PREPARATION OF A COMPOSITION (C1_{E}) ACCORDING TO THE INVENTION

The procedure of the process described in Example 1 is performed, replacing the molar equivalent of alanine with a molar equivalent of isoleucine and the 0.8 molar equivalent of octanoyl chloride with 0.8 molar equivalent of cocoyl chloride, to obtain composition (C1_{E}), the analytical characteristics of which are as follows:

Acid number (according to NFT 60-204)=68.0
pH 5% of composition (C1_{E}) in water (according to method NFT 73-206)=5.2
Hydroxyl number (according to U.S. Pharmacopea XXI NF XVI 01/011985)=188.5
Ester number (calculated by the difference between the saponification number measured according to method NFT 60-110 and the acid number measured according to method NFT 60-204)=54.7
Saponification number (according to NFT 60-206)=122.7 mg KOH/g

Said cocoyl chloride used in the preparation of Composition (C1_{E}) comprises, per 100% of its mass, 8% by mass of octanoyl chloride, 8% by mass of decanoyl chloride, 50% by mass of lauroyl chloride, 17% by mass of myristoyl chloride, 8% by mass of palmitoyl chloride, 3% by mass of stearoyl chloride, 4% by mass of oleoyl chloride and 2% by mass of linoleoyl chloride.

### Demonstration of the Antiaging Activity of the Compounds and Compositions According to the Invention Via an In Vitro Study of Migration of Human Fibroblasts.

Demonstration of the antiaging activity of the compositions according to the invention was performed by using a study model consisting in studying the migratory capacity of normal human fibroblasts not treated or treated with the compositions according to the invention and references. The fibroblast migration test is a test commonly used in the cosmetics and pharmaceutical sector and is especially described in the international patent application published under the number WO 2010/056908 A1. It allows in vitro reproduction of the phenomenon of fibroblast migration; a decrease in the migratory capacities of the fibroblasts being reported as associated with aging of the skin (1) (4).

### Protocol:

Normal human fibroblasts at the R5 passage were amplified in a T75 culture flask and then inoculated at 25 000 cells/well in specific culture plates. These plates are dedicated to migration (Oris™ from Platypus), integrating a central zone without initial deposition of cells (stoppers). After 96 hours of plate amplification and 2 hours before the treatment, all the wells were treated with mitomycin C (2 hours at 10 μg/ml), so as to stop the cell proliferation, and thus to observe only the migration phenomenon. The stoppers were removed, and the references, the products or standard medium for fibroblasts containing 2% fetal calf serum (FCS) were applied to the cells under 100 μl, and then incubated for 40 hours at 37° C. under 5% CO₂. Each condition was performed in quadruplicate.

### Evaluation of the Effects:

On conclusion of the incubation, the cells were labeled with Calcein AM (5 μM, 20 minutes at 37° C.), so as to visualize the viable cells by fluorescence (revelation of the cytoplasm). Photos were taken, after having inserted a black screen under the culture plates, so as to visualize only the zone where the cells were not deposited. Thus, only the cells which had migrated were photographed (×4 objective lens; with a ×0.7 reducer on the camera adapter). The mean migration distance of the cells (D_{migr}) was measured with NIS-Elements-BR 3.0 (4 cells). The means and standard deviations of the distances were calculated on the quadruplicates. The percentages of migratory effect relative to the control, and also the statistics (Student test) were also calculated.

### Results:

The results obtained are collated in Table 1 below:

TABLE 1
Migration of fibroblasts in the presence of the test compositions

| | | [(D_{migr}) − |
|---|---|---|
| | | (D_{migr})_{control}]/ |
| Test products (w/v) | (D_{migr}) in μm | (D_{migr})_{control} |
| FCS culture control (20 mg/ml) | 168 ± 34 | |
| Epidermal Growth Factor (EGF) 10 ng/ml | 203 ± 20 | +21% |
| Epidermal Growth Factor (EGF) 50 ng/ml | 212 ± 8 | +26% |
| Composition (C1_{A}) (5 μg/ml) | 206 ± 35 | +23% |
| Composition (C1_{A}) (10 μg/ml) | 188 ± 23 | +12% |
| Composition (C1_{B}) (5 μg/ml) | 190 ± 23 | +13% |
| Composition (C1_{B}) (10 μg/ml) | 195 ± 13 | +16% |
| Composition (C1_{C}) (5 μg/ml) | 198 ± 17 | +18% |
| Composition (C1_{C}) (10 μg/ml) | 188 ± 9 | +12% |
| Composition (C1_{D}) (5 μg/ml) | 181 ± 11 | +8% |
| Composition (C1_{D}) (10 μg/ml) | 188 ± 15 | +12% |
| Composition (C1_{E}) (5 μg/ml) | 197 ± 3 | +17% |
| Composition (C1_{E}) (10 μg/ml) | 192 ± 9 | +14% |

The combination of EGF at 10 and 50 ng/ml with the normal human fibroblasts show an increase in the migration distance of said fibroblasts, thus validating the chosen model.

The combination of compositions (C1_{A}), (C1_{B}), (C1_{C}), (C1_{D}) and (C1_{D}) with the normal human fibroblasts show a significant increase in the migration distance of said fibroblasts, and consequently an improvement in the migratory properties of the fibroblasts of human skin dermis, thus constituting an effective means for preventing and/or treating aging of the skin of the human body and the lips.

### BIBLIOGRAPHY

(1): Schulze et al., “Stiffening of human skin fibroblasts with age”; Clin. Plast. Surg.; 2012; 39(1):9-20.
(2): Baraibar and Friguet, “Oxidative proteome modifications target specific cellular pathways during oxidative stress, cellular senescence and ageing”; Exp Gerontol; 2013; 48(7):620-5.
(3): Kondo et al., “Inhibitory effects of human serum on human fetal skin fibroblast migration: migration-inhibitory activity and substances in serum, and its age-related changes”; In Vitro Cell Dev Biol Anim; 2000; 36(4):256-61.
(4): Jang et al., Prolonged activation of ERK contributes to the photorejuvenation effect in photodynamis therapy in human dermal fibroblasts; JID; 2013; 133(9):2265-75.
(5): Houreld and Abrahamse “Low-intensity laser irradiation stimulates wound healing in diabetic wounded fibroblast cells (WS1)”, 2010, Diabetes Technol Ther, December; 12(12)
(6): Tang et al., “A rice-derived recombinant human lactoferrin stimulates fibroblast proliferation, migration, and sustains cell survival”, 2010, Wound Repair Regen, January-February 18(1)
(7): Demirovic and Rattan, “Curcumin induces stress response and hormetically modulates wound healing ability of human skin fibroblasts undergoing ageing in vitro”, 2011, Biogerontology, March 6
(8): Rojo et al., “Wound healing properties of nut oil from Pouteria lucuma”, 2010, J Cosmet Dermatol, September 9(3)
9: Karleskind A., 1992. Manuel des corps gras [Handbook of fats], Lavoisier, Vol. 1 and 2: 65-78, 115-241, 1072-1089, 1433-1459.

1. A cosmetic composition comprising a compound of formula (I): 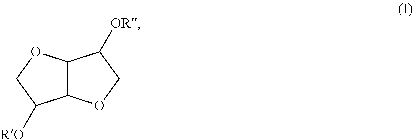
in which R′ and R″, which may be identical or different, represent either a hydrogen atom; or a monovalent radical of formula (IIa): 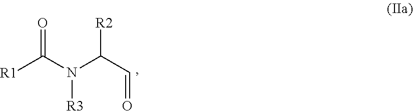
in which formula (IIa) the group R1-C(═O)— is chosen from octanoyl, decanoyl, ω-undecylenoyl, dodecanoyl, tetradecanoyl, hexadecanoyl, octadecanoyl, 9-octadecenoyl or 9,12-octadecadienoyl radicals, R3 represents a hydrogen atom, and R2 represents a radical chosen from methyl, isopropyl, isobutyl and 1-methylpropyl radicals, it being understood that, in said formula (I), at least one of the radicals R′ and R″ does not represent a hydrogen atom and that when none of the radicals R′ and R″ represents a hydrogen atom, R′ and R″ are identical, or of a mixture of compounds of formula (I).

2. The cosmetic composition of claim 1, comprising, per 100% of its mass:
from 99% by mass to 20% by mass of at least one compound of formula (Ia) 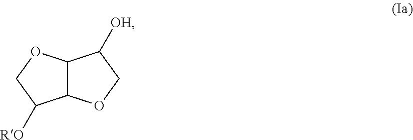
in which formula (Ia) R′ represents either a monovalent radical of formula (IIa): 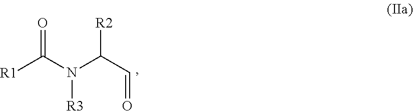
in which formula (IIa) the group R1-C(═O)— is chosen from octanoyl, decanoyl, ω-undecylenoyl, dodecanoyl, tetradecanoyl, hexadecanoyl, octadecanoyl, 9-octadecenoyl and 9,12-octadecadienoyl radicals, R3 represents a hydrogen atom and R2 represents a radical chosen from methyl, isopropyl, isobutyl and 1-methylpropyl radicals, and
from 1% by mass to 80% by mass of at least one compound of formula (Ib): 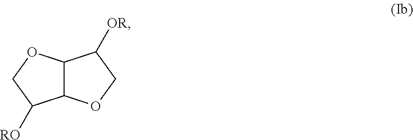
in which formula (Ib) R represents either a monovalent radical of formula (IIa): 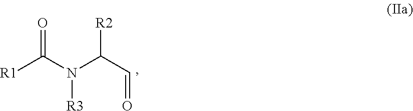
in which formula (IIa) the group R1-C(═O)— is chosen from octanoyl, decanoyl, ω-undecylenoyl, dodecanoyl, tetradecanoyl, hexadecanoyl, octadecanoyl, 9-octadecenoyl and 9,12-octadecadienoyl radicals, R3 represents a hydrogen atom and R2 represents a radical chosen from methyl, isopropyl, isobutyl and 1-methylpropyl radicals.

3. A process for the purpose of preventing or slowing the appearance of wrinkles or fine lines, impairment of the microrelief, lack of elasticity and/or tonicity, lack of density and/or firmness of human skin or the lips, or else for eliminating them, comprising at least one step of applying to human skin or to the lips a cosmetic formulation for topical use comprising at least one cosmetically acceptable excipient and an effective amount of at least one compound of formula (I): 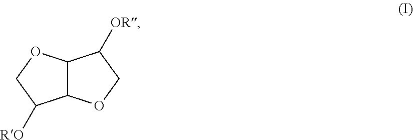
in which R′ and R″, which may be identical or different, represent either a hydrogen atom; or a monovalent radical of formula (IIa): 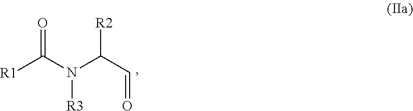
in which formula (IIa) the group R1-C(═O)— is chosen from octanoyl, decanoyl, ω-undecylenoyl, dodecanoyl, tetradecanoyl, hexadecanoyl, octadecanoyl, 9-octadecenoyl or 9,12-octadecadienoyl radicals, R3 represents a hydrogen atom, and R2 represents a radical chosen from methyl, isopropyl, isobutyl and 1-methylpropyl radicals, it being understood that, in said formula (I), at least one of the radicals R′ and R″ does not represent a hydrogen atom and that when none of the radicals R′ and R″ represents a hydrogen atom, R′ and R″ are identical.

4. A process for the purpose of preventing or slowing the appearance of wrinkles or fine lines, impairment of the microrelief, lack of elasticity and/or tonicity, lack of density and/or firmness of human skin or the lips, or else for eliminating them, comprising at least one step of applying to human skin or to the lips a cosmetic formulation for topical use comprising at least one cosmetically acceptable excipient and an effective amount of composition (C1) comprising, per 100% of its mass:
from 99% by mass to 20% by mass of at least one compound of formula (Ia) 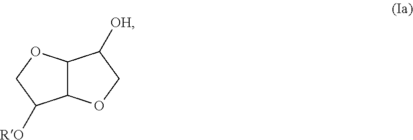
in which formula (Ia) R′ represents either a monovalent radical of formula (IIa): 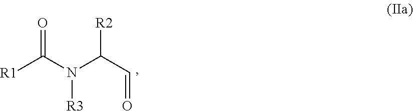
in which formula (IIa) the group R1-C(═O)— is chosen from octanoyl, decanoyl, ω-undecylenoyl, dodecanoyl, tetradecanoyl, hexadecanoyl, octadecanoyl, 9-octadecenoyl and 9,12-octadecadienoyl radicals, R3 represents a hydrogen atom and R2 represents a radical chosen from methyl, isopropyl, isobutyl and 1-methylpropyl radicals, and
from 1% by mass to 80% by mass of at least one compound of formula (Ib): 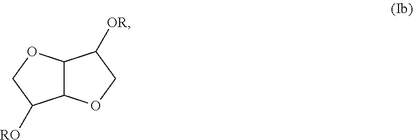
in which formula (Ib) R represents either a monovalent radical of formula (IIa): 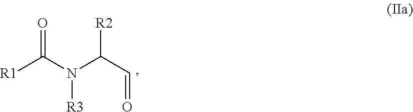
in which formula (IIa) the group R1-C(═O)— is chosen from octanoyl, decanoyl, ω-undecylenoyl, dodecanoyl, tetradecanoyl, hexadecanoyl, octadecanoyl, 9-octadecenoyl and 9,12-octadecadienoyl radicals, R3 represents a hydrogen atom and R2 represents a radical chosen from methyl, isopropyl, isobutyl and 1-methylpropyl radicals.
